(19) United States
(12) Patent Application Publication
(10) Pub. No.: US 2026/0055051 A1
(43) Pub. Date: Feb. 26, 2026

(54) Compound with anti-tumor activity, preparation method therefor, and use thereof

(71) Applicant: Ocean University of China, Qingdao, Shandong Province (CN)

(72) Inventors: Liangmin Yu, Qingdao, Shandong Province (CN); Xiaohui Jiang, Qingdao, Shandong Province (CN); Xuefeng Yan, Qingdao, Shandong Province (CN); Xia Li, Qingdao, Shandong Province (CN); Zhiming Zhang, Qingdao, Shandong Province (CN); Changcheng Li, Qingdao, Shandong Province (CN); Zhiyu He, Qingdao, Shandong Province (CN); Xuan Wang, Qingdao, Shandong Province (CN)

(21) Appl. No.: 19/101,882

(22) PCT Filed: Mar. 2, 2023
(86) PCT No.: PCT/CN2023/079217

§ 371 Date: Feb. 6, 2025

(30) Foreign Application Priority Data
Aug. 9, 2022 (CN) 202210948185.3

(51) Int. Cl. C07C 233/87; A61K 31/165; A61K 31/192; A61K 31/235; A61P 35/00; C07C 233/18; C07C 233/47; C07C 233/49

(52) U.S. Cl. CPC C07C 233/87; A61K 31/165; A61K 31/192; A61K 31/235; A61P 35/00; C07C 233/18; C07C 233/47; C07C 233/49

(57) ABSTRACT

The present invention relates to a compound of chemical formula (I), and further relates to a preparation method therefor and use thereof. Pharmacological experiment results show that the compound of the present invention has excellent anti-tumor activity, good stability, low toxicity, and broad-spectrum efficacy, and can be used as an anti-tumor medicament, and provides a theoretical basis for subsequent research and development of drugs.

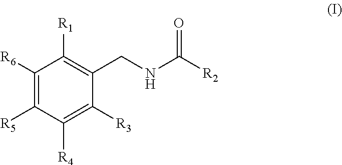

### RELATED APPLICATIONS

This application is the U.S. National Stage of International Application No. PCT/CN2023/079217, filed Mar. 2, 2023, which designates the U.S., published in Chinese, and claims priority under 35 U.S.C. § 119 or 365(c) to Chinese Application No. 202210948185.3, filed Aug. 9, 2022. The entire teachings of the above applications are incorporated herein by reference.

### TECHNICAL FIELD

The present disclosure belongs to the pharmaceutical field. More specifically, the present disclosure relates to a compound with anti-tumor activity, a method for preparing the same, and use thereof.

### BACKGROUND ART

Cancer, also known as malignant tumor, is among the major diseases that seriously threaten human life and health. According to the data from the Global Cancer Observatory (GCO) website in 2018, the global incidence and mortality of cancer were 10.8 million and 9.56 million cases, respectively.

Natural products harbor a variety of active ingredients with potential anti-cancer effects due to their rich sources and diverse structures, and have become a treasure trove of drugs for cancer treatment. Currently, numerous natural active ingredients have been shown to have anti-tumor activity, but there are only a few commercialized natural anti-cancer drugs with good activity. Therefore, developing natural products with excellent activity has become an urgent need for cancer treatment.

Capsaicin (compound name: trans-8-methyl-N-vanillyl-6-nonenamide) has the following chemical structure:

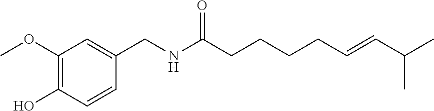 [SMILES: COc1cc(CNC(=O)CCCC/C=C/C(C)C)ccc1O]

Capsaicin is a vanillylamide-based alkaloid with various biological activities isolated from Capsicum species in the Solanaceae family. Capsaicin's anti-tumor activity has become a research hotspot in recent years. Many in vitro and in vivo studies have shown that capsaicin effectively inhibits the growth of various tumors, such as breast cancer, bladder cancer, liver cancer, prostate cancer, endometrial cancer, non-small-cell lung cancer, and colon cancer, and is a broad-spectrum anti-tumor compound.

However, it still does not meet the actual needs in cancer treatment, and presents certain technical problems. In view of the drawbacks in the prior art, the inventors completed the present disclosure after studying the previous work and carrying out extensive experimental research and analyses.

### SUMMARY OF DISCLOSURE

An object of embodiments according to the disclosure is to provide a compound with anti-tumor activity.

Another object of embodiments according to the disclosure is to provide a method for preparing the compound with anti-tumor activity.

A further object of embodiments according to the disclosure is to provide use of the compound with anti-tumor activity.

The present disclosure is achieved through the following technical solutions.

An embodiment according to the disclosure relates to a compound with anti-tumor activity.

The compound with anti-tumor activity has the following structural formula:

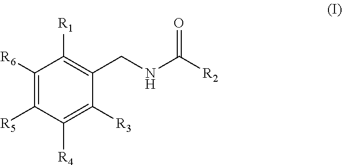

in the formula,
R₁ is selected from hydroxyl, carboxyl, methyl, methoxy, and hydrogen;
R₂ is selected from vinyl, methyl, monochloromethyl, and phenyl;
R₃ is selected from carbomethoxy, ethoxycarbonyl, propoxycarbonyl, hydroxyl, methyl, and hydrogen;
R₄ is selected from hydrogen, hydroxyl, chloro, methyl, bromo, acetyl, carbomethoxy, and chloroacetamidomethyl;
R₅ is selected from hydroxyl, methyl, and hydrogen;
R₆ is selected from hydroxyl, acrylamidomethyl, acetamidomethyl, chloroacetamidomethyl, methyl, and hydrogen.

According to an embodiment of the present disclosure, R₂ is vinyl; R₁ is carboxyl; and R₆ is acrylamidomethyl.

According to an embodiment of the present disclosure, R₂ is vinyl; R₁ is carboxyl; R₆ is acrylamidomethyl; and R₃ and R₅ are hydroxyl.

According to an embodiment of the present disclosure, R₂ is vinyl; R₁ is carboxyl; R₆ is acrylamidomethyl; R₃ and R₅ are hydroxyl; and R₄ is hydroxyl or bromo.

According to an embodiment of the present disclosure, R₂ is methyl; R₁ is methyl; and R₆ is hydrogen.

According to an embodiment of the present disclosure, R₂ is methyl; R₁ is methyl; R₆ is hydrogen; and R₃ and R₅ are each hydroxyl or methyl.

According to an embodiment of the present disclosure, R₂ is methyl; R₁ is methyl; R₆ is hydrogen; R₃ and R₅ are each hydroxyl or methyl; and R₄ is methyl.

According to an embodiment of the present disclosure, R₂ is monochloromethyl; R₃ is carbomethoxy, hydroxyl, or methyl.

According to an embodiment of the present disclosure, R₂ is monochloromethyl; R₃ is carbomethoxy, hydroxyl, or methyl; and R₆ is selected from hydroxyl, chloroacetamidomethyl, and hydrogen.

According to an embodiment of the present disclosure, R₂ is monochloromethyl; R₃ is carbomethoxy, hydroxyl, or methyl; R₆ is selected from hydroxyl, chloroacetamidomethyl, and hydrogen; and R₅ is hydroxyl or methyl.

According to an embodiment of the present disclosure, R₂ is monochloromethyl; R₃ is carbomethoxy, hydroxyl, or methyl; R₆ is selected from hydroxyl, chloroacetamidomethyl, and hydrogen; R₅ is hydroxyl or methyl; and R₄ is selected from hydrogen, chlorine, and methyl.

According to an embodiment of the present disclosure, R₂ is monochloromethyl; R₃ is carbomethoxy, hydroxyl, or methyl; R₆ is selected from hydroxyl, chloroacetamidomethyl, and hydrogen; R₅ is hydroxyl or methyl; R₄ is selected from hydrogen, chlorine, and methyl; and R₁ is selected from hydroxyl, methyl, methoxy, and hydrogen.

According to an embodiment of the present disclosure, the compound is selected from the following compounds:

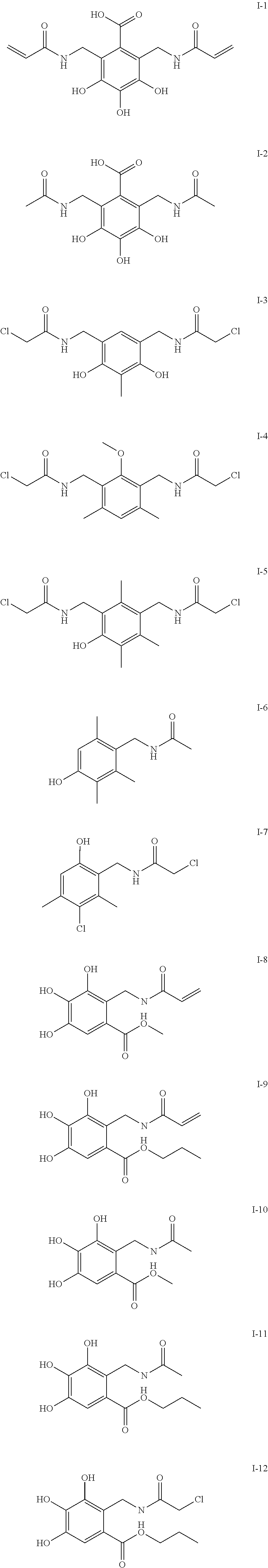

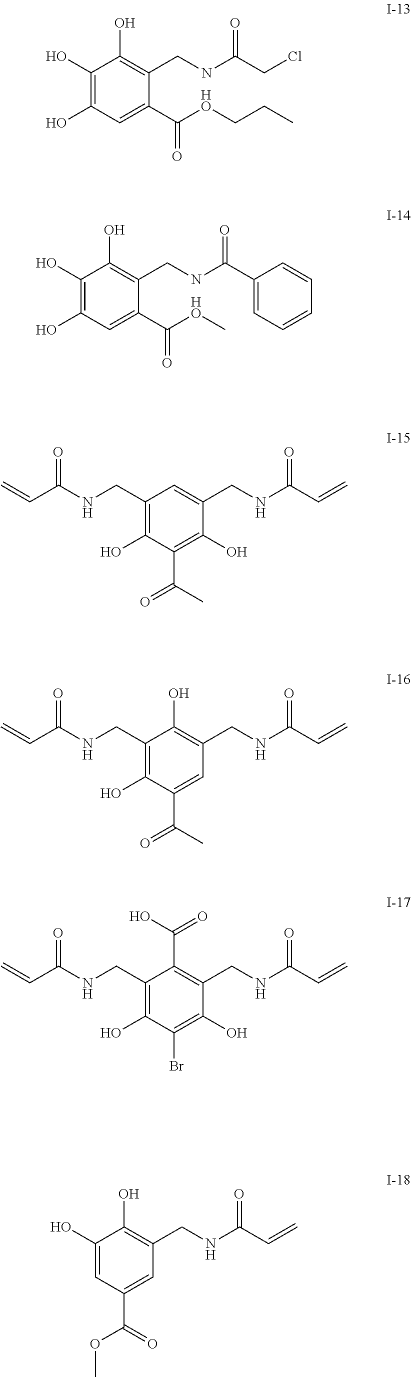

An embodiment according to the disclosure relates to a method for preparing the compound.

The preparation steps of the method are as follows:
allowing an aromatic compound (represented by formula (II) below) and an amide compound (represented by formula (III) below) in a molar ratio of 1:1.2˜2.4 to undergo a substitution reaction in an organic solvent at 25 to 55° C. for 48 to 96 hours in the presence of a catalyst, in which the molar ratio of the aromatic compound to the catalyst is 1:1.8˜1.9;
then carrying out filtration to obtain a solid product, and
thoroughly washing the solid product with deionized water and drying it to obtain the compound of chemical formula (I).

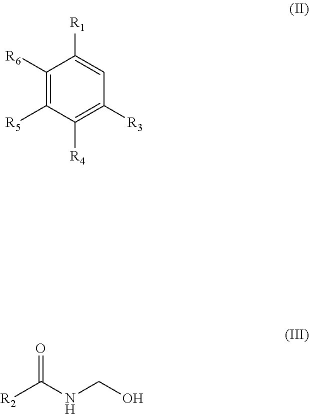

According to another embodiment of the present disclosure, the aromatic compound is gallic acid, methyl gallate, propyl gallate, 2,6-dihydroxytoluene, 3,5-dimethylanisole, 2,3,5-trimethylphenol, 4-chloro-3,5-dimethylphenol, 2,6-dihydroxyacetophenone, 2,4-dihydroxyacetophenone, 4-bromo-3,5-dihydroxybenzoic acid, or methyl 3,4-dihydroxybenzoate.

According to another embodiment of the present disclosure, the amide compound is N-hydroxymethyl acrylamide, N-hydroxymethyl acetamide, N-hydroxymethyl chloroacetamide, or N-hydroxymethyl benzamide.

According to another embodiment of the present disclosure, the catalyst is concentrated sulfuric acid or anhydrous aluminum trichloride.

According to another embodiment of the present disclosure, the solvent is dichloromethane, trichloromethane, acetone, or ethanol.

According to another embodiment of the present disclosure, the washed solid product is dried at a temperature of 50 to 60° C. for 360 to 420 min, to obtain a dried solid product having a water content of 5% by weight or less.

An embodiment according to the disclosure relates to use of the compound of chemical formula (I), or a compound of chemical formula (I) obtained by the above method, as an anti-tumor drug.

According to an embodiment of the present disclosure, the compound of chemical formula (I) is for use in the preparation of an anti-tumor drug for the treatment of lung cancer, liver cancer, colon cancer, leukemia, cervical cancer, or breast cancer.

A more detailed description of the disclosure is provided below.

An embodiment according to the disclosure relates to a compound with anti-tumor activity.

The compound with anti-tumor activity has the following structural formula:

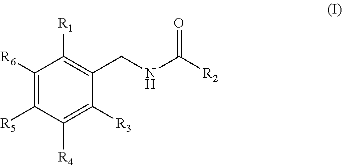

in the formula,
R₁ is selected from hydroxyl, carboxyl, methyl, methoxy, and hydrogen;
R₂ is selected from vinyl, methyl, monochloromethyl, and phenyl;
R₃ is selected from carbomethoxy, ethoxycarbonyl, propoxycarbonyl, hydroxyl, methyl, and hydrogen;
R₄ is selected from hydrogen, hydroxyl, chloro, methyl, bromo, acetyl, carbomethoxy, and chloroacetamidomethyl;
R₅ is selected from hydroxyl, methyl, and hydrogen;
R₆ is selected from hydroxyl, acrylamidomethyl, acetamidomethyl, chloroacetamidomethyl, methyl, and hydrogen.

According to an embodiment of the present disclosure, the compound is selected from the following compounds:

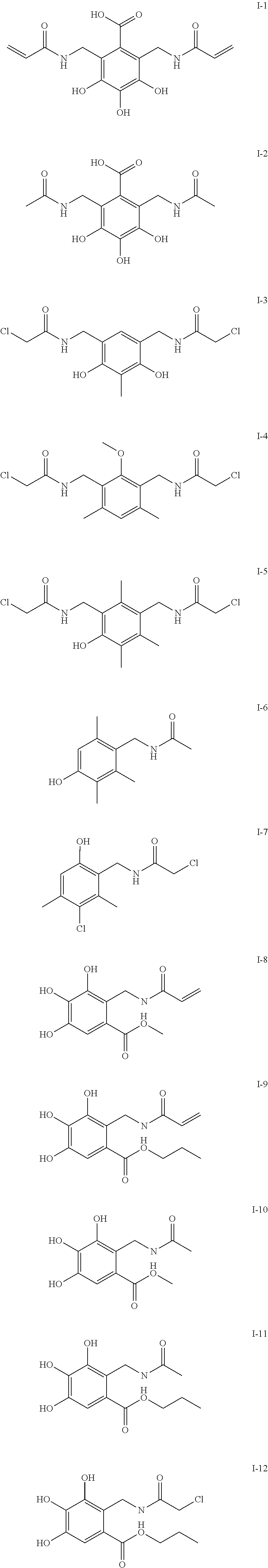

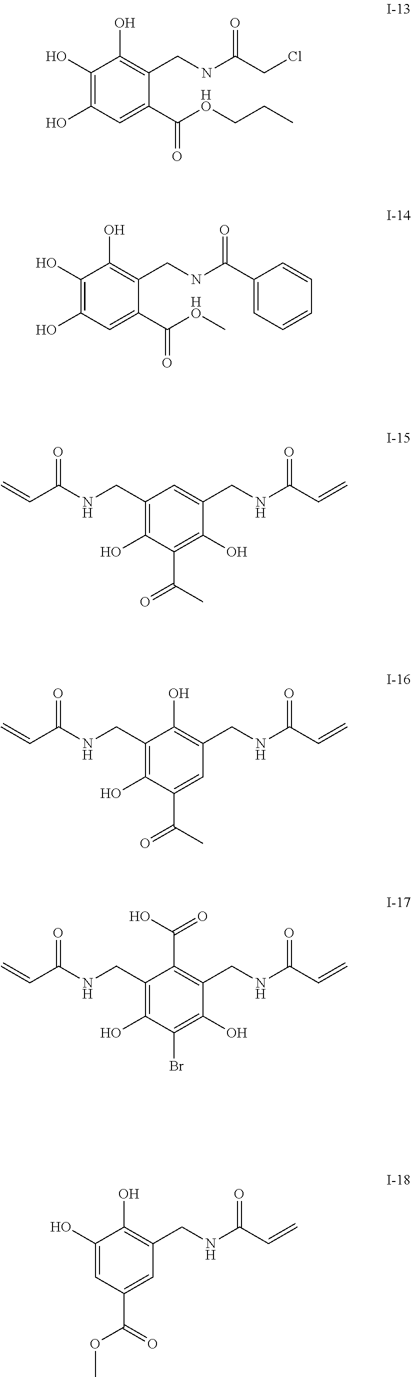

An embodiment according to the disclosure relates to a method for preparing the compound.

The preparation steps of the compound preparation method are as follows:
allowing an aromatic compound (represented by formula (II) below) and an amide compound (represented by formula (III) below) in a molar ratio of 1:1.2˜2.4 to undergo a substitution reaction in an organic solvent at 25 to 55° C. for 48 to 96 hours in the presence of a catalyst, in which the molar ratio of the aromatic compound to the catalyst is 1:1.8˜1.9;
then carrying out filtration to obtain a solid product, and
thoroughly washing the solid product with deionized water and drying it to obtain the compound of chemical formula (I).

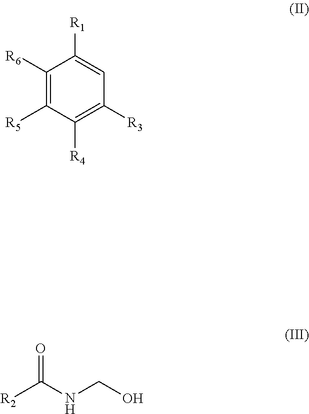

According to the present disclosure, the aromatic compound may be gallic acid, methyl gallate, propyl gallate, 2,6-dihydroxytoluene, 3,5-dimethylanisole, 2,3,5-trimethylphenol, 4-chloro-3,5-dimethylphenol, 2,6-dihydroxyacetophenone, 2,4-dihydroxyacetophenone, 4-bromo-3,5-dihydroxybenzoic acid, or methyl 3,4-dihydroxybenzoate.

The aromatic compounds used according to the present disclosure may be commercially available products, for example, gallic acid from Sinopharm Chemical Reagent Co. Ltd. under the trade name Gallic acid, methyl gallate from Sinopharm Chemical Reagent Co. Ltd. under the trade name Methyl gallate, 2,6-dihydroxytoluene from Sinopharm Chemical Reagent Co. Ltd. under the trade name 2,6-Dihydroxytoluene, 3,5-dimethylanisole from Sinopharm Chemical Reagent Co. Ltd. under the trade name 3,5-Dimethylanisole, 4-chloro-3,5-dimethylphenol from Sinopharm Chemical Reagent Co. Ltd. under the trade name 4-Chloro-3,5-dimethylphenol, 2,6-dihydroxyacetophenone from Sinopharm Chemical Reagent Co. Ltd. under the trade name 2,6-Dihydroxyacetophenone, 2,4-dihydroxyacetophenone from Sinopharm Chemical Reagent Co. Ltd. under the trade name 2,4-Dihydroxyacetophenone, 4-bromo-3,5-dihydroxybenzoic acid from Sinopharm Chemical Reagent Co. Ltd. under the trade name 4-Bromo-3,5-dihydroxybenzoic acid, and methyl 3,4-dihydroxybenzoate from Sinopharm Chemical Reagent Co. Ltd. under the trade name Methyl 3,4-dihydroxybenzoate.

According to the present disclosure, the amide compound may be N-hydroxymethyl acrylamide (abbreviated as N-1), N-hydroxymethyl acetamide (abbreviated as N-2), N-hydroxymethyl chloroacetamide (abbreviated as N-3), or N-hydroxymethyl benzamide (abbreviated as N-4). The amide compounds used according to the present disclosure are commercially available products, for example, N-hydroxymethyl acrylamide from Sinopharm Chemical Reagent Co. Ltd. under the trade name N-hydroxymethyl acrylamide, N-hydroxymethyl acetamide from Shanghai Abotchem Co. Ltd. under the trade name N-hydroxymethyl acetamide, N-hydroxymethyl chloroacetamide from Shanghai Abotchem Co. Ltd. under the trade name Chloroacetamide-N-methanol, or N-hydroxymethyl benzamide from Sinopharm Chemical Reagent Co. Ltd. under the trade name N-Hydroxymethyl benzamide.

According to the present disclosure, the catalyst may be concentrated sulfuric acid or anhydrous aluminum trichloride. Both are commercially available chemical products commonly used in the field of chemical engineering.

According to the present disclosure, the solvent may be dichloromethane, trichloromethane, acetone, or ethanol. All of them are commercially available chemical products commonly used in the field of chemical engineering.

In the present disclosure, the molar ratio of the aromatic compound to the amide compound is 1:1.2˜2.4. If the molar ratio of the aromatic compound to the amide compound is greater than 1:1.2, the aromatic compound cannot react completely, which adversely affects product purification and cannot achieve a desired yield. If the molar ratio of the aromatic compound to the amide compound is smaller than 1:2.4, the yield will not be further improved and the amide compound will be wasted. Therefore, it is reasonable to set the molar ratio of the aromatic compound to the amide compound to 1:1.2˜2.4.

In the present disclosure, the molar ratio of the aromatic compound to the catalyst is 1:1.8˜1.9. If the molar ratio of the aromatic compound to the catalyst is greater than 1:1.8, the reaction will be incomplete and thus a desired yield cannot be achieved. If the molar ratio of the aromatic compound to the catalyst is smaller than 1:1.9, the yield will be slightly decreased, which may be caused by oxidation of a part of the product by excess concentrated sulfuric acid. Therefore, an appropriate molar ratio of the aromatic compound to the catalyst is 1:1.8˜1.9.

The aromatic compound and the amide compound are subjected to a substitution reaction in an organic solvent at a temperature of 25 to 55° C. for 48 to 96 hours in the presence of a catalyst. In the present disclosure, when the substitution reaction duration is within the above range, if the substitution reaction temperature is lower than 25° C., the reaction duration will be too long, and a desired yield will not even be achieved; and if the substitution reaction temperature is higher than 55° C., side reactions occur, unfavorable to obtainment of the desired compound. Therefore, a substitution reaction temperature of 25 to 55° C. is appropriate, and it is preferably 35 to 40° C. Similarly, when the substitution reaction temperature is within the above range, if the substitution reaction duration is shorter than 48 h, the substitution reaction will be incomplete, and a desired yield will not be achieved; and if the substitution reaction duration is longer than 96 h, the yield will not be further improved, and energy and time will be wasted. Therefore, it is appropriate to set the substitution reaction duration to 48 to 96 h, preferably 60 to 84 h;

According to the present disclosure, the washed solid product is dried at a temperature of 50 to 60° C. for 360 to 420 min, to obtain a dried solid product having a water content of 5% by weight or less. In the present disclosure, the water content of the dried solid product is measured according to the standard method of GB 5009.3-85. It is undesirable for the water content of the dried solid product to exceed the above range, as it will result in inaccurate dosing of the compound in subsequent anti-tumor experiments, which in turn affects the measurement results of the anti-tumor activity of the compound.

The yield of the method for preparing the compound is calculated according to the following equation:

$$Yield ( \% ) = {m}_{product} / ( {M}_{product} \times {n}_{aromatic compound} ) \times 100 \%$$

in the equation,
m_{product} is the mass of the compound of formula (I) in grams;
M_{product} is the molar mass of the compound of formula (I) in g/mol;
n_{aromatic compound} is the number of moles of the aromatic compound;

In the synthesis experiment, because the amide compound is in excess, the number of moles of the product is equal to the number of moles of the aromatic compound.

An embodiment according to the present disclosure relates to use of the above compound or a compound of chemical formula (I) obtained by the above method as an anti-tumor drug.

According to the present disclosure, the compound of chemical formula (I) is for use in the preparation of an anti-tumor drug for the treatment of lung cancer, liver cancer, colon cancer, leukemia, cervical cancer or breast cancer.

Compared to the prior art, the present disclosure has the following beneficial effects.

The preparation method according to the disclosure uses mild reaction conditions, reagents with low toxicity, readily available raw materials, and simple post-processing, and has a high yield. Pharmacological experiments showed that the compound of the present disclosure exhibited excellent anti-tumor activity, good stability, low toxicity, and broad-spectrum activity, and can be used as an anti-tumor drug, providing a theoretical basis for future drug development.

### BRIEF DESCRIPTION OF DRAWINGS

FIG. 1 shows the inhibitory effects of different concentrations of a compound of chemical formula (I) on tumor cell lines;

FIG. 2 is a graph showing the average body weight trend of mice transplanted with a tumor cell line after administration with a compound of chemical formula (I);

FIG. 3 is a graph showing the average tumor volume growth trend of mice transplanted with a tumor cell line after administration with a compound of chemical formula (I);

FIG. 4 is a statistical graph showing the average tumor weight of mice transplanted with a tumor cell line after administration with a compound of chemical formula (I);

FIG. 5 shows photographs of tumors of mice transplanted with a tumor cell line after administration with a compound of chemical formula (I);

FIG. 6 is a statistical graph showing the inhibition rate of average tumor weight in mice transplanted with a tumor cell line after administration with a compound of chemical formula (I).

### DESCRIPTION OF EMBODIMENTS

The technical solutions of the present disclosure will be described in detail with reference to the accompanying drawings and examples; but the scope of the disclosure is not limited thereto.

### Example 1: Preparation of N-(2,3,4-trihydroxy-5-acrylamidomethyl-6-carboxybenzyl) acrylamide

The procedure of the example was as follows.

An aromatic compound gallic acid and an amide compound N-hydroxymethyl acrylamide in a molar ratio of 1:2.4 were allowed to undergo a substitution reaction in anhydrous ethanol as an organic solvent at 35 to 40° C. for 72 hours in the presence of concentrated sulfuric acid as a catalyst, with the molar ratio of the aromatic compound to the catalyst being 1:1.9. Then the reaction solution was filtered, and the resultant solid was thoroughly washed with deionized water and dried at a temperature of 55° C. for 400 min. As measured according to the method described in the specification of this application, the dried product had a water content of 4.5% by weight and was white solid powder. The yield calculated according to the method described in the specification of the application was 84.26%.

This dried product was subjected to conventional IR, ¹H NMR, ¹³C NMR, and HR-ESI-MS analyses, and the results are as follows:

IR (KBr) v: 819.05, 985.89, 1118.34, 1588.12, 1618.13, 1656.18, 1711.79, 3142.38, 3313.61, 3441.67 cm⁻¹.

¹H NMR (DMSO, 600 MHz) δ: 4.59 (s, 2H, CH₂), 4.67 (s, 1H, CH₂), 4.68 (s, 12H, CH₂), 5.67 (m, 1H, =CH), 5.95 (m, 1H, =CH), 6.18 (m, 1H, =CH₂), 6.38 (d, J=6.00 Hz, 1H, =CH₂), 6.42 (m, 1H, =CH₂), 7.66 (m, 1H, =CH₂), 9.07 (t, J=6.00 Hz, 1H, NH), 9.47 (s, 1H, OH), 9.52 (s, 1H, OH), 10.43 (s, 1H, OH).

¹³C NMR (DMSO, 150 MHz) δ: 32.89, 45.52, 116.01, 118.13, 122.56, 127.32, 130.05, 130.68, 140.19, 141.07, 146.17, 165.16, 167.48, 168.47.

HR-ESI-MS: m/z 337.1036 ([M+H]+, calculated for C₁₅H₁₇N₂O₇: 337.3117), 359.0852 ([M+Na]+, calculated for C₁₅H₁₆N₂O₇Na: 359.3038).

It can be seen that the product was N-(2,3,4-trihydroxy-5-acrylamidomethyl-6-carboxybenzyl) acrylamide (I-1) of the following chemical structure formula:

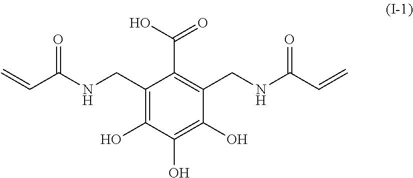 [SMILES: C=CC(=O)NCc1c(O)c(O)c(O)c(CNC(=O)C=C)c1C(=O)O]

### Example 2: Preparation of N-(2,3,4-trihydroxy-5-acetamidomethyl-6-carboxybenzyl) acetamide

The same procedure as in Example 1 was employed, except that N-hydroxymethyl acetamide was used instead of N-hydroxymethyl acrylamide. A white solid product was obtained with a yield of 65.37%.

The resulting dried product was subjected to conventional IR, ¹H NMR, ¹³C NMR and HR-ESI-MS analyses, and the results are as follows:

IR (KBr) v: 815.39, 934.66, 1122.73, 1570.56, 1600.56, 1677.40, 1707.40, 3112.38, 3334.83, 3441.67 cm⁻¹.

¹H NMR (DMSO, 600 MHz) δ: 1.89 (s, 3H, CH₃), 4.56 (s, 1H, CH₂), 4.57 (s, 1H, CH₂), 4.49 (s, 2H, CH₂), 8.96 (t, J=6.00 Hz, 1H, NH), 9.38 (s, 1H, OH), 9.43 (s, 1H, OH), 10.60 (s, 1H, OH), 12.39 (s, 1H, COOH).

¹³C NMR (DMSO, 150 MHz) δ: 22.08, 24.93, 32.88, 45.20, 116.15, 118.13, 122.15, 140.11, 140.81, 146.12, 168.44, 170.27, 173.29.

HR-ESI-MS: m/z 314.0673 ([M+H]⁺, calculated for C₁₃H₁₇N₂O₇: 313.2897), 335.0809 ([M+Na]⁺, calculated for C₁₃H₁₆N₂O₇Na: 335.2818).

It can be seen that the product was N-(2,3,4-trihydroxy-5-acetamidomethyl-6-carboxybenzyl) acetamide (I-2) of the following chemical structure formula:

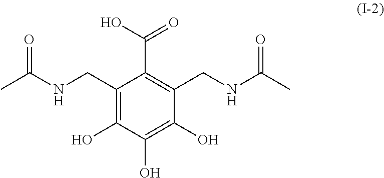 [SMILES: CC(=O)NCc1c(O)c(O)c(O)c(CNC(C)=O)c1C(=O)O]

### Example 3: Preparation of N-(2,4-dihydroxy-3-methyl-5-chloroacetamidomethylbenzyl) chloroacetamide

The same procedure as in Example 1 was employed, except that 2,6-dihydroxytoluene was used instead of gallic acid, and N-hydroxymethyl chloroacetamide was used instead of N-hydroxymethyl acrylamide. An off-white solid product was obtained with a yield of 66.25%.

The resulting dried product was subjected to conventional IR, ¹H NMR, ¹³C NMR and HR-ESI-MS analyses, and the results are as follows:

IR (KBr) v: 778.50, 1123.23, 1262.88, 1449.61, 1543.78, 1625.17, 2950.64, 3154.92, 3324.10 cm⁻¹.

¹H NMR (DMSO, 600 MHz) δ: 2.02 (s, 3H, CH₃), 4.13 (s, 4H, CH₂), 4.15 (s, 2H, CH₂), 4.15 (s, 2H, CH₂), 6.80 (s, 1H, PhH), 8.76 (t, J=6.00 Hz, 2H, NH), 8.89 (s, 2H, OH).

¹³C NMR (DMSO, 150 MHz) δ: 10.01, 39.41, 42.84, 113.11, 116.54, 128.58, 153.85, 167.59.

HR-ESI-MS: m/z 358.0407 ([M+Na]⁺, calculated for C₁₃H₁₆N₂O₄Cl₂Na: 358.1818).

It can be seen that the product was N-(2,4-dihydroxy-3-methyl-5-chloroacetamidomethylbenzyl) chloroacetamide (I-3) of the following chemical structure:

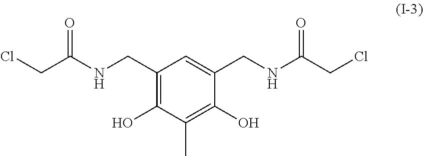 [SMILES: Cc1c(O)c(CNC(=O)CCl)cc(CNC(=O)CCl)c1O]

### Example 4: Preparation of N-(2-methoxy-3-chloroacetamidomethyl-4,6-dimethylbenzyl) chloroacetamide

The same procedure as in Example 1 was employed, except that 3,5-dimethylanisole was used instead of gallic acid, and N-hydroxymethyl chloroacetamide was used instead of N-hydroxymethyl acrylamide. A white solid product was obtained with a yield of 58.29%.

The resulting dried product was subjected to conventional IR, ¹H NMR, ¹³C NMR and HR-ESI-MS analyses, and the results are as follows:

IR (KBr) v: 591.13, 1052.83, 1140.20, 1389.50, 1462.56, 1543.15, 1638.05, 2831.83, 2956.10, 3285.24 cm⁻¹.

¹H NMR (DMSO, 600 MHz) δ: 2.24 (t, J=6.00 Hz, 3H, CH₃), 2.27 (s, 3H, CH₃), 3.70 (s, 2H, CH₂), 3.77 (d, J=12.00 Hz, 2H, CH₂), 4.02 (t, J=6.00 Hz, 3H, CH₃), 4.24 (d, J=6.00 Hz, 1H, CH₂), 4.26 (d, J=6.00 Hz, 1H, CH₂), 4.28 (d, J=6.00 Hz, 1H, CH₂), 4.32 (d, J=6.00 Hz, 1H, CH₂), 6.625 (s, 1H, PhH), 8.06 (t, J=6.00 Hz, 1H, NH), 8.18 (d, J=6.00 Hz, 1H, NH).

¹³C NMR (DMSO, 150.92 MHz) δ: 15.56, 20.12, 34.86, 37.59, 42.99, 43.01, 55.37, 109.87, 111.06, 113.75, 123.51, 126.85, 139.16, 158.66, 166.02.

HR-ESI-MS: m/z 370.0778 ([M+Na]⁺, calculated for C₁₅H₂₀N₂O₃Cl₂Na: 370.2352).

It can be seen that the product was N-(2-methoxy-3-chloroacetamidomethyl-4,6-dimethylbenzyl) chloroacetamide (I-4) of the following chemical structure:

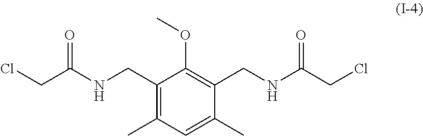 [SMILES: COc1c(CNC(=O)CCl)c(C)cc(C)c1CNC(=O)CCl]

### Example 5: Preparation of N-(2,3,6-trimethyl-4-hydroxy-5-chloroacetamidomethylbenzyl) chloroacetamide

The same procedure as in Example 1 was employed, except that 2,3,5-trimethylphenol was used instead of gallic acid, and N-hydroxymethyl chloroacetamide was used instead of N-hydroxymethyl acrylamide. A white solid product was obtained with a yield of 43.29%.

The resulting dried product was subjected to conventional IR, ¹H NMR, ¹³C NMR and HR-ESI-MS analyses, and the results are as follows:

IR (KBr) v: 780.93, 1101.03, 1232.09, 1405.32, 1455.78, 1543.90, 1641.06, 2919.20, 3054.77, 3269.42 cm⁻¹.

¹H NMR (DMSO, 600 MHz) δ: 2.11 (s, 3H, CH₃), 2.16 (s, 3H, CH₃), 2.27 (s, 3H, CH₃), 4.04 (s, 2H, CH₂), 4.17 (s, 2H, CH₂), 4.26 (s, 1H, CH₂), 4.27 (s, 1H, CH₂), 4.28 (s, 1H, CH₂), 4.29 (s, 1H, CH₂), 8.24 (t, 1H, NH), 9.32 (s, 1H, OH), 9.37 (t, 1H, NH).

¹³C NMR (DMSO, 150 MHz) δ: 13.23, 15.92, 16.42, 36.74, 38.72, 45.52, 42.99, 121.84, 122.48, 126.20, 134.93, 137.09, 153.43, 165.97, 168.36.

HR-ESI-MS: m/z 370.0776 ([M+Na]⁺, calculated for C₁₅H₂₀N₂O₃Cl₂Na: 370.2352).

It can be seen that the product was N-(2,3,6-trimethyl-4-hydroxy-5-chloroacetamidomethylbenzyl) chloroacetamide (I-5) of the following chemical structure formula:

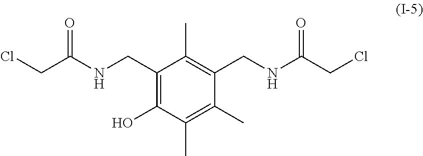 [SMILES: Cc1c(C)c(CNC(=O)CCl)c(C)c(CNC(=O)CCl)c1O]

### Example 6: Preparation of N-(2,3,6-trimethyl-4-hydroxybenzyl) acetamide

The same procedure as in Example 1 was employed, except that 2,3,5-trimethylphenol was used instead of gallic acid, N-hydroxymethyl acetamide was used instead of N-hydroxymethyl acrylamide, and the molar ratio of 2,3,5-trimethylphenol to N-hydroxymethyl acetamide was 1:1.2. A white solid product was obtained with a yield of 56.35%.

The resulting dried product was subjected to conventional IR, ¹H NMR, ¹³C NMR and HR-ESI-MS analyses, and the results are as follows:

IR (KBr) v: 852.49, 1088.98, 1308.16, 1556.70, 1464.06, 1417.37, 1624.49, 2855.93, 2927.48, 3303.31 cm⁻¹.

¹H NMR (DMSO, 600 MHz) δ: 1.78 (s, 3H, CH₃), 2.02 (s, 3H, CH₃), 2.12 (s, 3H, CH₃), 2.17 (s, 3H, CH₃), 4.14 (s, 1H, CH₂), 4.15 (s, 1H, CH₂), 6.53 (s, 1H, PhH), 7.70 (s, 1H, NH), 9.12 (s, 1H, OH).

¹³C NMR (DMSO, 150 MHz) δ: 12.36, 16.05, 20.10, 22.80, 37.83, 114.51, 120.35, 125.62, 134.91, 137.33, 154.50, 169.17.

HR-ESI-MS: m/z 208.1333 ([M+H]⁺, calculated for C₁₂H₁₈NO₂: 208.2798), 230.1153 ([M+Na]⁺, calculated for C₁₂H₁₇NO₂Na: 230.2720).

It can be seen that the product was N-(2,3,6-trimethyl-4-hydroxybenzyl) acetamide (I-6) of the following chemical structure formula:

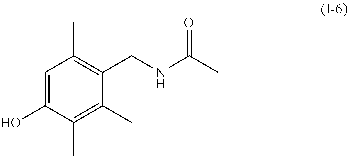

### Example 7: Preparation of N-(2,4-dimethyl-3-chloro-6-hydroxybenzyl) chloroacetamide

The same procedure as in Example 1 was employed, except that 4-chloro-3,5-dimethylphenol was used instead of gallic acid, N-hydroxymethyl chloroacetamide was used instead of N-hydroxymethyl acrylamide, and the molar ratio of 4-chloro-3,5-dimethylphenol to N-hydroxymethyl chloroacetamide was 1:1.2. A white solid product was obtained with a yield of 79.26%.

The resulting dried product was subjected to conventional IR, ¹H NMR, ¹³C NMR and HR-ESI-MS analyses, and the results are as follows:

IR (KBr) v: 844.20, 1067.89, 1168.82, 1543.90, 1455.78, 1400.80, 1637.29, 2936.52, 3277.71, 3358.30 cm⁻¹.

¹H NMR (DMSO, 600 MHz) δ: 2.24 (s, 3H, CH₃), 2.29 (s, 3H, CH₃), 4.04 (s, 1H, CH₂), 4.31 (d, J=6.00 Hz, 1H, CH₂), 6.70 (s, 1H, PhH), 8.25 (s, 1H, NH), 9.71 (s, 1H, OH).

¹³C NMR (DMSO, 150.92 MHz) δ: 17.03, 21.11, 35.77, 42.93, 115.77, 122.40, 124.56, 135.98, 136.33, 154.67, 166.30.

HR-ESI-MS: m/z 262.0403 ([M+H]⁺, calculated for C₁₁H₁₄NO₂Cl₂: 263.1375), 284.0221 ([M+Na]⁺, calculated for C₁₁H₁₃NO₂Cl₂Na: 285.1296).

It can be seen that the product was N-(2,4-dimethyl-3-chloro-6-hydroxybenzyl) chloroacetamide (I-7) of the following chemical structure formula:

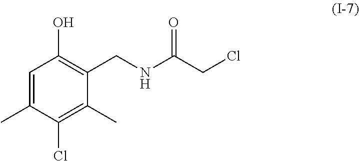 [SMILES: Cc1cc(O)c(CNC(=O)CCl)c(C)c1Cl]

### Example 8: Preparation of N-(2,3,4-trihydroxy-6-carbomethoxy-benzyl) acrylamide

The same procedure as in Example 1 was employed, except that methyl gallate was used instead of gallic acid, and the molar ratio of methyl gallate to N-hydroxymethyl acrylamide was 1:1.2. A white solid product was obtained with a yield of 93.27%.

The resulting dried product was subjected to conventional IR, ¹H NMR, ¹³C NMR and HR-ESI-MS analyses, and the results are as follows:

IR (KBr) v: 633.91, 1100.04, 1217.85, 1442.50, 1538.37, 1593.98, 1689.84, 2964.56, 3183.36, 3402.89 cm⁻¹.

¹H NMR (DMSO, 600 MHz) δ: 3.78 (s, 3H, CH₃), 4.51 (d, 2H, CH₂), 5.64 (m, 1H, CH═), 6.15 (m, 1H, =CH₂), 6.43 (m, 1H, =CH₂), 6.98 (s, 1H, PhH), 8.67 (t, J=6.00 Hz, 1H, NH), 9.04 (s, 1H, OH), 9.27 (s, 1H, OH), 10.18 (s, 1H, OH).

¹³C NMR (DMSO, 150.92 MHz) δ: 36.03, 52.21, 110.55, 118.92, 119.70, 126.90, 131.05, 139.10, 144.96, 145.71, 166.96, 167.41.

HR-ESI-MS: m/z 268.0813 ([M+H]⁺, calculated for C₁₂H₁₄NO₆: 268.2427), 290.0633 ([M+Na]⁺, calculated for C₁₂H₁₃NO₆Na: 290.2247).

It can be seen that the product is N-(2,3,4-trihydroxy-6-carbomethoxy-benzyl) acrylamide (I-8) of the following chemical structure formula:

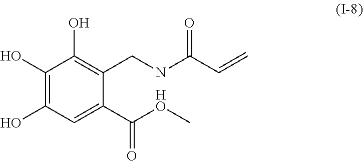

### Example 9: Preparation of (2,3,4-trihydroxy-6-propoxycarbonyl-benzyl) acrylamide

The same procedure as in Example 1 was employed, except that propyl gallate was used instead of gallic acid, and the molar ratio of propyl gallate to N-hydroxymethyl acrylamide was 1:1.2. A white solid product was obtained with a yield of 85.92%.

The resulting dried product was subjected to conventional IR, ¹H NMR, ¹³C NMR and HR-ESI-MS analyses, and the results are as follows:

IR (KBr) v: 637.57, 1089.80, 1230.29, 1442.50, 1541.29, 1600.56, 1689.83, 2971.15, 3370.68 cm⁻¹.

¹H NMR (DMSO, 600 MHz) δ: 0.97 (t, J=6.00 Hz, 3H, CH₃), 1.70 (d, 2H, CH₂), 4.16 (t, J=6.00 Hz, 2H, CH₂), 4.53 (d, J=6.00 Hz, 2H, CH₂), 5.64 (m, 1H, CH═), 6.15 (m, 1H, =CH₂), 6.44 (m, 1H, =CH₂), 7.02 (s, 1H, PhH), 8.64 (t, J=6.00 Hz, 1H, NH), 9.05 (s, 1H, OH), 9.29 (s, 1H, OH), 10.10 (s, 1H, OH).

¹³C NMR (DMSO, 150.92 MHz) δ: 10.96, 22.09, 36.01, 66.13, 110.43, 118.88, 119.96, 126.74, 131.13, 138.99, 144.92, 145.73, 166.85, 166.95.

HR-ESI-MS: m/z 296.1130 ([M+H]⁺, calculated for C₁₄H₁₈NO₆: 296.2976), 318.0950 ([M+Na]⁺, calculated for C₁₄H₁₇NO₆Na: 318.2796).

It can be seen that the product was N-(2,3,4-trihydroxy-6-propoxycarbonyl-benzyl) acrylamide (I-9) of the following chemical structure formula:

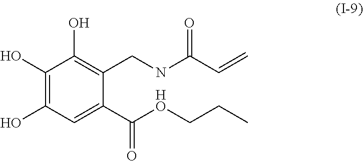

### Example 10: Preparation of N-(2,3,4-trihydroxy-6-carbomethoxy-benzyl) acetamide

The same procedure as in Example 1 was employed, except that methyl gallate was used instead of gallic acid, N-hydroxymethyl acetamide was used instead of N-hydroxymethyl acrylamide, and the molar ratio of methyl gallate to N-hydroxymethyl acetamide was 1:1.2. A white solid product was obtained with a yield of 91.36%.

The resulting dried product was subjected to conventional IR, ¹H NMR, ¹³C NMR and HR-ESI-MS analyses, and the results are as follows:

IR (KBr) v: 686.9, 1043.79, 1214.76, 1540.89, 1595.12, 1626.00, 1688.51, 2953.84, 3233.27, 3380.89 cm⁻¹.

¹H NMR (DMSO, 600 MHz) δ: 1.90 (s, 3H, CH₃), 3.79 (s, 3H, CH₃), 4.42 (d, 2H, CH₂), 6.97 (s, 1H, PhH), 8.54 (t, J=6.00 Hz, 1H, NH), 9.00 (s, 1H, OH), 9.20 (s, 1H, OH), 10.37 (s, 1H, OH).

¹³C NMR (DMSO, 150.92 MHz) δ: 22.20, 36.08, 52.19, 110.60, 119.27, 119.53, 139.22, 144.88, 145.66, 167.43, 172.73.

HR-ESI-MS: m/z 256.0816 ([M+H]⁺, calculated for C₁₁H₁₄NO₆: 256.2319), 278.0634 ([M+Na]⁺, calculated for C₁₁H₁₃NO₆Na: 278.2139).

It can be seen that the product was N-(2,3,4-trihydroxy-6-carbomethoxy-benzyl) acetamide (I-10) of the following chemical structure formula:

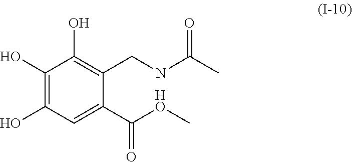

### Example 11: Preparation of N-(2,3,4-trihydroxy-6-propoxycarbonyl-benzyl) acetamide

The same procedure as in Example 1 was employed, except that propyl gallate was used instead of gallic acid, N-hydroxymethyl acetamide was used instead of N-hydroxymethyl acrylamide, and the molar ratio of propyl gallate to N-hydroxymethyl acetamide was 1:1.2. A white solid product was obtained with a yield of 89.68%.

The resulting dried product was subjected to conventional IR, ¹H NMR, ¹³C NMR and HR-ESI-MS analyses, and the results are as follows:

IR (KBr) v: 722.94, 1110.82, 1213.26, 1550.68, 1579.30, 1623.74, 1689.26, 2970.41, 3281.21, 3402.73 cm⁻¹.

¹H NMR (DMSO, 600 MHz) δ: 0.97 (t, J=6.00 Hz, 3H, CH₃), 1.70 (m, 2H, CH₂), 1.88 (s, 3H, CH₃), 4.15 (t, J=6.00 Hz, 2H, CH₂), 4.40 (d, J=6.00 Hz, 2H, CH₂), 6.99 (s, 1H, PhH), 8.49 (t, J=6.00 Hz, 1H, NH), 8.99 (s, 1H, OH), 9.20 (s, 1H, OH), 10.28 (s, 1H, OH).

¹³C NMR (DMSO, 150.92 MHz) δ: 10.96, 22.11, 36.02, 66.09, 100.00, 110.47, 119.30, 119.76, 139.12, 144.84, 145.68, 166.95, 172.58.

HR-ESI-MS: m/z 284.1130 ([M+H]⁺, calculated for C₁₃H₁₈NO₆: 284.2868), 306.0947 ([M+Na]⁺, calculated for C₁₃H₁₇NO₆Na: 306.2688).

It can be seen that the product was N-(2,3,4-trihydroxy-6-propoxycarbonyl-benzyl) acetamide (I-11) of the following chemical structure formula:

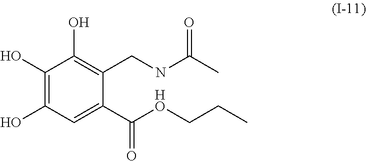

### Example 12: Preparation of N-(2,3,4-trihydroxy-6-carbomethoxy-benzyl) chloroacetamide

The same procedure as in example 1 was employed, except that methyl gallate was used instead of gallic acid, N-hydroxymethyl chloroacetamide was used instead of N-hydroxymethyl acrylamide, and the molar ratio of methyl gallate to N-hydroxymethyl chloroacetamide was 1:1.2. A white solid product was obtained with a yield of 58.52%.

The resulting dried product was subjected to conventional IR, ¹H NMR, ¹³C NMR and HR-ESI-MS analyses, and the results are as follows:

IR (KBr) v: 767.38, 1052.83, 1213.26, 1543.15, 1594.36, 1638.05, 1689.36, 2948.57, 3402.73 cm⁻¹.

¹H NMR (DMSO, 600 MHz) δ: 3.77 (s, 3H, CH₃), 4.12 (s, 2H, CH₂), 4.50 (d, J=6.00 Hz, 2H, CH₂), 6.97 (s, 1H, PhH), 8.36 (t, J=6.00 Hz, 1H, NH), 9.18 (s, 1H, OH), 9.32 (s, 1H, OH), 9.43 (s, 1H, OH).

¹³C NMR (DMSO, 150.92 MHz) δ: 35.94, 42.75, 52.23, 110.23, 118.15, 120.12, 138.53, 144.96, 145.64, 167.27, 167.49.

HR-ESI-MS: m/z 290.0427 ([M+H]⁺, calculated for C₁₁H₁₃NO₆Cl:290.6767), 312.0244 ([M+Na]⁺, calculated for C₁₁H₁₂NO₆ClNa: 312.6587).

It can be seen that the product was N-(2,3,4-trihydroxy-6-carbomethoxy-benzyl) chloroacetamide (I-12) of the following chemical structure formula:

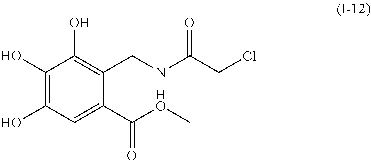

### Example 13: Preparation of N-(2,3,4-trihydroxy-6-propoxycarbonyl-benzyl) chloroacetamide

The same procedure as in Example 1 was employed, except that propyl gallate was used instead of gallic acid, N-hydroxymethyl chloroacetamide was used instead of N-hydroxymethyl acrylamide, and the molar ratio of propyl gallate to N-hydroxymethyl chloroacetamide was 1:1.2. A white solid product was obtained with a yield of 43.18%.

The resulting dried product was subjected to conventional IR, ¹H NMR, ¹³C NMR and HR-ESI-MS analyses, and the results are as follows:

IR (KBr) v: 657.41, 1045.30, 1242.83, 1543.15, 1586.83, 1638.05, 1668.48, 2963.63, 3182.81, 3410.26 cm⁻¹.

¹H NMR (DMSO, 600 MHz) δ: 0.96 (t, J=6.00 Hz, 3H, CH₃), 1.70 (m, 2H, CH₂), 4.11 (s, 2H, CH₂), 4.14 (t, J=6.00 Hz, 2H, CH₂), 4.49 (d, J=6.00 Hz, 2H, CH₂), 7.00 (s, 1H, PhH), 8.32 (t, J=6.00 Hz, 1H, NH), 9.18 (s, 1H, OH), 9.27 (s, 1H, OH), 9.44 (s, 1H, OH).

¹³C NMR (DMSO, 150.92 MHz) δ: 10.96, 22.07, 36.98, 42.76, 66.23, 110.14, 118.06, 120.41, 138.42, 144.93, 146.86, 167.09, 167.11.

HR-ESI-MS: m/z 318.0738 ([M+H]⁺, calculated for C₁₃H₁₇NO₆Cl:318.7316), 340.0555 ([M+Na]⁺, calculated for C₁₃H₁₆NO₆ClNa: 340.7136).

It can be seen that the product was N-(2,3,4-trihydroxy-6-propoxycarbonyl-benzyl) chloroacetamide (I-13) of the following chemical structure formula:

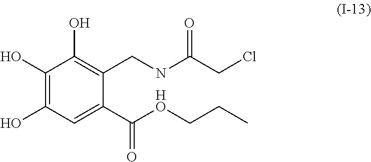

### Example 14: Preparation of N-(2,3,4-trihydroxy-6-carbomethoxy-benzyl) benzamide

The same procedure as in Example 1 was employed, except that methyl gallate was used instead of gallic acid, N-hydroxymethyl benzamide was used instead of N-hydroxymethyl acrylamide, and the molar ratio of methyl gallate to N-hydroxymethyl benzamide was 1:1.2. A white solid product was obtained with a yield of 83.14%.

The resulting dried product was subjected to conventional IR, ¹H NMR, ¹³C NMR and HR-ESI-MS analyses, and the results are as follows:

IR (KBr) v: 3443.00, 3227.54, 2957.02, 1687.42, 1591.66, 1531.81, 1490.31, 1252.51, 1096.10, 725.03 cm⁻¹.

¹H NMR (DMSO, 600 MHz) δ: 3.77 (d, J=18.00 Hz, 3H, CH₃), 4.68 (s, 1H, CH₂), 4.69 (s, 1H, CH₂), 6.96 (s, 1H, PhH), 7.45 (t, J=6.00 Hz, 2H, PhH), 7.53 (t, J=6.00 Hz, 1H, PhH), 7.83 (s, 1H, PhH), 7.85 (s, 1H, PhH), 8.63 (t, J=6.00 Hz, 1H, NH), 9.08 (s, 1H, OH), 9.33 (s, 1H, OH), 9.78 (s, 1H, OH).

¹³C NMR (DMSO, 150.92 MHz) δ: 52.25, 36.30, 108.96, 110.43, 118.58, 120.43, 127.94, 128.76, 131.98, 134.00, 138.64, 144.87, 145.77, 146.05, 153.50, 167.73, 168.11.

HR-ESI-MS: m/z 318.0974 ([M+H]⁺, calculated for C₁₆H₁₆NO₆: 318.0978), 340.0789 ([M+Na]⁺, calculated for C₁₆H₁₅NO₆Na: 340.2921).

It can be seen that the product was N-(2,3,4-trihydroxy-6-carbomethoxy-benzyl) benzamide (I-14) of the following chemical structure formula:

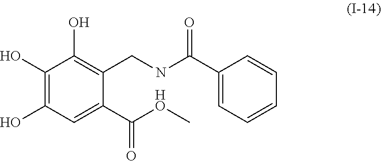

### Experimental Example 1: In Vitro Tumor Cell Proliferation Inhibition Assay

The procedure of this experimental example was as follows.

### I. Test Methods

### Sulforhodamine B Colorimetric Method (SRB Method) and MTT Colorimetric Method (MTT Method)

### II. Test Samples

The compounds prepared according to the present disclosure; the existing doxorubicin as a positive control;

### III. Apparatus

Ultra-clean bench (SW-CJ-2F) from Suzhou Antai Airtech Co., Ltd.; Milli-Q Ultrapure water system (AdvantageA 5) from Millipore, USA; microscope (CX41) from Olympus, Japan; CO₂ cell incubator (Heracell150i) from Thermo; electric constant temperature water bath (HWS-24) from Shanghai Yiheng Technology Instrument Co., Ltd.; multimode Microplate Reader (SpectraMax® 13) from Molecular Devices, USA; automatic cell counter (Muse™ cell analyzer) from Millipore, USA.

### IV. Experimental Materials and Reagents

96-well cell culture plates and 25 cm² culture flasks from ExCell Bio (Taicang) Co., Ltd. PBS phosphate buffer from Solarbio life sciences; Fetal bovine serum (FBS) (FND500) from ExCell Bio (Taicang) Co., Ltd.; L-glutamine (G8230) and Penicillin-Streptomycin Liquid (100×) (P1400) from Solarbio life sciences; RPMI.1640 culture medium (1×) (GNM31800) and DMEM high glucose culture medium (1×) (GNM12800) from GENOM Biopharmaceutical Technology Co., Ltd.; Gibco 0.05% trypsin-EDTA (25300-054) from Invitrogen, USA; Tris (T8060) and SDS (S8010) from Solarbio life sciences; SRB (S1402) from Sigma life science, and MTT from Solarbio life sciences.

### V. Test Cell Lines

Human lung cancer cell line A549, human liver cancer cell line HepG2, human colon cancer cell line HCT116, human colon cancer cell line HT-29, human leukemia cell line K562, human cervical cancer cell line hela, and human breast cancer cell line MCF-7, all provided by the Shanghai Cell Bank, Chinese Academy of Sciences.

### VI. Test Process

A549, HepG2, HCT116, HT-29, K562, hela, and MCF-7 cells were cultured respectively in 1640, DMEM, 5A, 5A, 1640, DMEM, and 1640 culture media containing 10 wt % heat-inactivated FBS (fetal bovine serum), 2 mM L-glutamine, 100 U/mL penicillin and 100 mg/mL streptomycin, in a cell incubator at 37° C. and 5 vol % CO₂. The medium was replaced every two days. After the A549, HepG2, HCT116, HT-29, hela, and MCF-7 cells were confluent, they were digested with 0.05% (w/v) trypsin-EDTA at 37° C., passaged, and maintained in the logarithmic growth phase suitable for testing. The K562 suspension cells were passaged without digestion, and maintained in the logarithmic growth phase suitable for testing. The A549, HepG2, HCT116, HT-29, K562, hela, and MCF-7 cells in the logarithmic growth phase were inoculated to 96-well plates at 5000, 5000, 5000, 5000, 6000, 5000, 5000 per well, respectively, and cultured at 37° C. for 24 h.

Then various concentrations of test samples were added. The positive control group was doxorubicin hydrochloride (final concentration was 1 μM), the solvent control group was an equal volume of DMSO, and the blank control group was an equal volume of a culture medium, with 4 replicate wells for each concentration. After the A549, HepG2, HCT116, HT-29, hela, and MCF-7 cells were treated with the test drugs for 72 hours, the cells in each well were fixed with 50% (m/v) cold trichloroacetic acid (TCA) and stained with SRB. Then 150 mL/well of Tris solution was added, and the OD value at the wavelength of 540 nm was measured using a microplate reader. For K562 cells, after 72 hours of treatment with the test drugs, 20 mL of 5 mg/mL MTT was added, and the cells were incubated at 37° C. for 4 hours. Next, 100 mL of a triplet solution (10% SDS, 5% isobutanol, 12 mM HCl) was added, followed by further incubation for 12 to 20 hours. The OD value at the wavelength of 570 nm was measured using a microplate reader.

The tumor cell growth inhibition rate was calculated according to the following equation:

$$Inhibition \text{} rate = [ \text{} ( {OD}_{540 control well} - {OD}_{540 dosing well} ) / {OD}_{540 control well} ] \times 100 \%$$

in the equation,
OD_{540 control well} is the absorbance of the control group;
OD_{540 dosing well} is the absorbance of the test group.

IC50 values of the test drugs (calculated by GraphPad Prism 5 software) were the means of the results of triplicate experiments.

### VII. Test Results

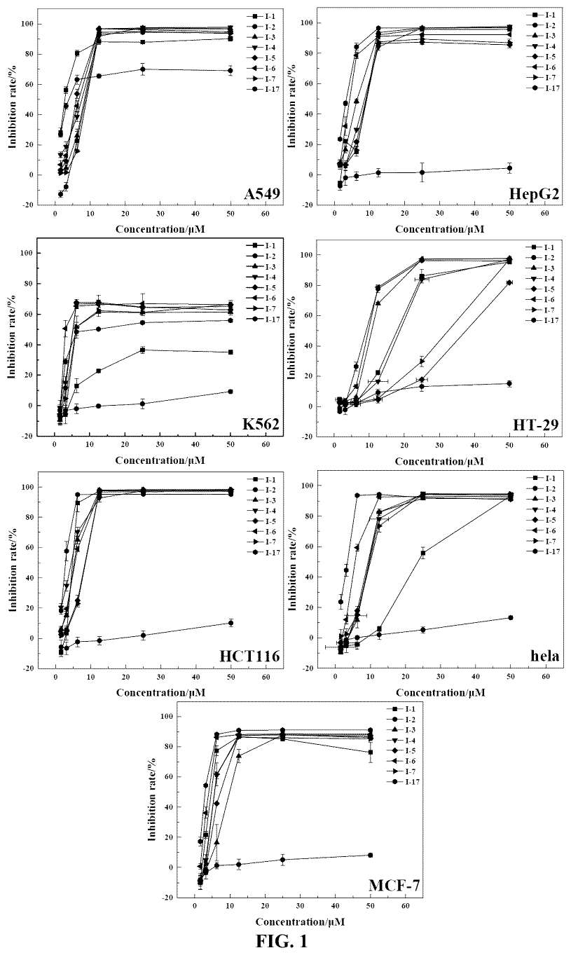

The test results are listed in Table 1 to Table 3 and FIG. 1.

TABLE 1
Inhibition rate on A549, HepG2 and HCT116
cell lines by test compounds (10 μM)

| | Inhibition rate/% | | |
|---|---|---|---|
| Compound | A549 | HepG2 | HCT116 |
| I-1 | 89.29 | 57.55 | 92.28 |
| I-2 | 69.87 | 18.41 | 82.00 |
| I-3 | 95.86 | 92.27 | 76.50 |
| I-4 | 68.99 | 82.86 | 69.33 |
| I-5 | 89.47 | 95.26 | 97.35 |
| I-6 | 94.94 | 81.09 | 96.64 |
| I-7 | 55.02 | 95.84 | 95.06 |
| I-8 | 28.52 | 54.82 | 30.36 |
| I-9 | 27.46 | 51.14 | 29.07 |
| I-10 | 38.18 | 55.75 | 34.76 |
| I-11 | 32.49 | 55.46 | 38.22 |
| I-12 | 40.93 | 55.93 | 47.61 |
| I-13 | 29.63 | 55.01 | 37.77 |
| I-14 | 37.69 | 1.56 | 34.07 |
| I-15 | 7.05 | 33.54 | 27.73 |
| I-16 | 4.84 | 12.96 | 33.88 |
| I-17 | 95.77 | 90.29 | 91.51 |
| I-18 | 10.46 | 73.04 | 22.50 |

TABLE 2
IC₅₀ values of certain test compounds against A549, HepG2,
HCT116, HT-29, K562, hela, and MCF-7 cell lines.

IC₅₀/μM

| Compound | I-1 | I-2 | I-3 | I-4 |
|---|---|---|---|---|
| A549 | 2.73 | 4.84 | 7.40 | 6.57 |
| HepG2 | 8.31 | >50 | 6.04 | 7.90 |
| HCT116 | 4.77 | >50 | 5.12 | 3.96 |
| HT-29 | 16.61 | >50 | 10.74 | 17.64 |
| K562 | >50 | >50 | 14.53 | 9.36 |
| hela | 23.78 | >50 | 9.30 | 9.38 |
| MCF-7 | 4.60 | >50 | 9.73 | 5.75 |

TABLE 3
IC₅₀ values of certain test compounds against A549, HepG2,
HCT116, HT-29, K562, hela, and MCF-7 cell lines.

IC₅₀/μM

| Compound | I-5 | I-6 | I-7 | I-17 |
|---|---|---|---|---|
| A549 | 6.32 | 5.95 | 8.11 | 7.77 |
| HepG2 | 4.06 | 8.36 | 8.61 | 3.05 |
| HCT116 | 5.32 | 7.36 | 7.44 | 2.74 |
| HT-29 | 9.39 | 35.42 | 28.92 | 8.60 |
| K562 | 6.93 | 9.74 | 12.74 | 18.02 |
| hela | 5.64 | 8.86 | 9.68 | 3.01 |
| MCF-7 | 3.74 | 6.98 | 5.75 | 2.93 |

The inhibition rates by doxorubicin (1 μM) on A549, HepG2, HCT116, HT-29, K562, hela, and MCF-7 cell lines were measured in the same manner as described above, and were 88.81%, 78.10%, 60.92%, 95.34%, 40.75%, 44.26%, and 69.08%, respectively.

The data in Table 1 to table 3 and the data of doxorubicin indicate that, although the anti-tumor effects of the compounds of the present disclosure were weaker than those of doxorubicin, most of the compounds still exhibited good inhibitory activity against tumor cells, and were closely related to the concentration of the compounds. As the concentration increased, the inhibitory effect was gradually enhanced, as shown in FIG. 1. Beyond 12.5 μm, the inhibition rate by the compounds of the present disclosure remained basically constant and did not reach 100%, indicating that the compounds inhibit tumor cells rather than kill cells, suggesting a favorable safety profile. In addition, compounds I-3, I-4, I-5, I-6, I-7, and I-17 showed broad-spectrum activity and exhibited good inhibitory effects on all 7 types of cells, with an IC₅₀ value lower than 35.50 μM. Among them, compound I-5 showed the best anticancer effect, with an IC₅₀ value lower than 9.40 M on all 7 types of cells.

### Test Example 2: Anti-Tumor Efficacy Test for the Compounds of the Present Disclosure

The procedure of this test example is as follows.

BALB/c nude mice were subcutaneously inoculated with A549, HCT116 and HT-29 cells to establish tumor transplantation models.
Solvent Control Group: 2% DMSO+normal saline+sodium carbonate (the molar ratio of the compound to sodium carbonate is 1:2)
5-fluorouracil control group: 5-fluorouracil injection (20 mg/kg);

Test compound group: 1 mg of a test compound was fully dissolved in 40 L DMSO, and then diluted with normal saline to 0.5 mg/mL. 2 mL was used each time, freshly prepared before use on the same day.

### Test Methods:

Eight mice were in each group.

The solvent control group and the test compound group were administered at a dose of 10 mL/kg via tail vein injection (the dose for each administration was calculated based on the body weight on that day), 5 times a week, for a total of 15 times. The animals were euthanized after the last administration.

The 5-fluorouracil control group was administered twice a week for a total of 6 times.

The body weight and tumor diameter were measured twice a week during the administration period. Tumors and the spleen were removed and weighed after euthanasia. The tumor volume, relative tumor volume (RTV), relative tumor growth rate (T/C %), tumor volume inhibition rate (IR_{TV}%), and tumor weight inhibition rate (IR_{TW}%) were calculated. Data for animals that died accidentally were only listed but excluded from statistical analysis.

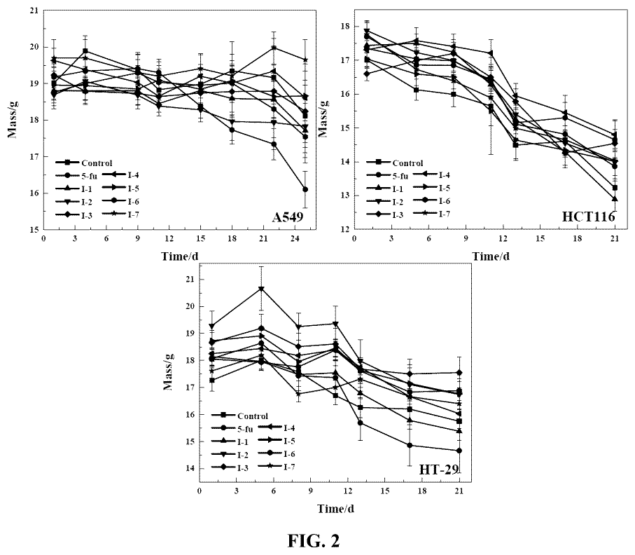

Throughout the experiment, the animals remained in a good condition. All groups showed a certain body weight gain, but a slight downward trend in body weight was seen near the end of study due to tumor growth. Compared to the solvent control, except for some compounds that caused a significantly lower weight on certain days, most treatment groups did not exhibit a significantly lower body weight. The significant weight loss of the animals in the 5-fu control group might be caused by the side effects of the chemotherapy drugs. The results are shown in FIG. 2.

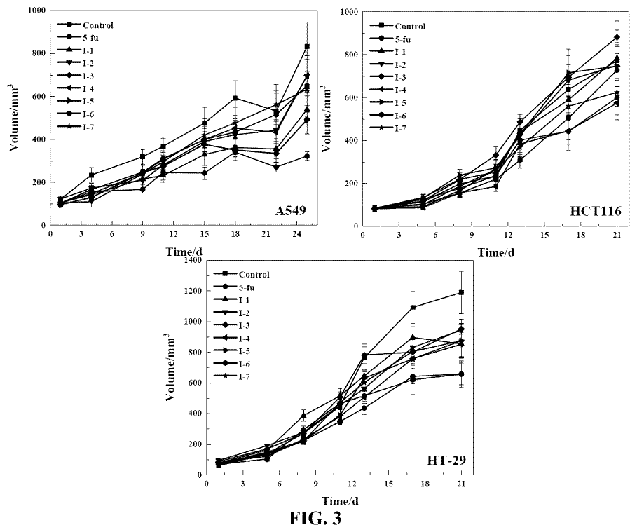

On the premise of confirming that the tumor cells grew normally in the mice, the tumor volume and weight were measured, and photographs thereof were taken. The results are shown in FIGS. 3 to 5. As shown in FIG. 3, the tumors in the solvent control group showed stable growth.

At the end of the experiment, the average tumor volumes of A549, HCT116 and HT-29 were 831.80, 771.76 and 1188.29 mm³, respectively. The tumors in the 5-fu group grew relatively slowly after administration, and the tumor volumes measured in the experiment were significantly lower than that in the solvent control group. At the end of the experiment, the average tumor volumes of A549, HCT116, and HT-29 were 322.03, 599.83, and 658.02 mm³, respectively.

In A549 cell-BALB/c nude mouse subcutaneous tumor transplantation experiment, the volumes of all test groups were larger than that of the control group. Among them, the volumes of the test groups with compounds I-1, I-6 and I-7 were relatively small, which were 539.48, 492.12 and 559.37 mm³, respectively.

In HCT116 cell-BALB/c nude mouse subcutaneous tumor transplantation experiment, the volume of the test group with compound I-4, 574.36 mm³, was smaller than that of the control group. Furthermore, the volume of the test group with compound I-7, 624.48 mm³, was slightly larger than that of the control group.

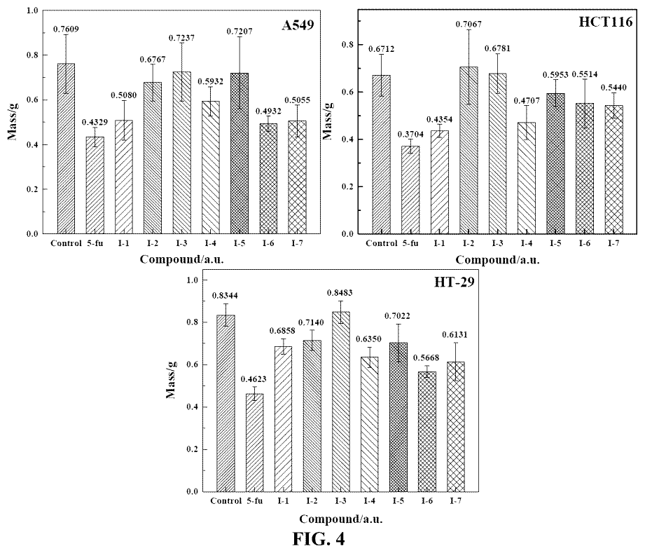

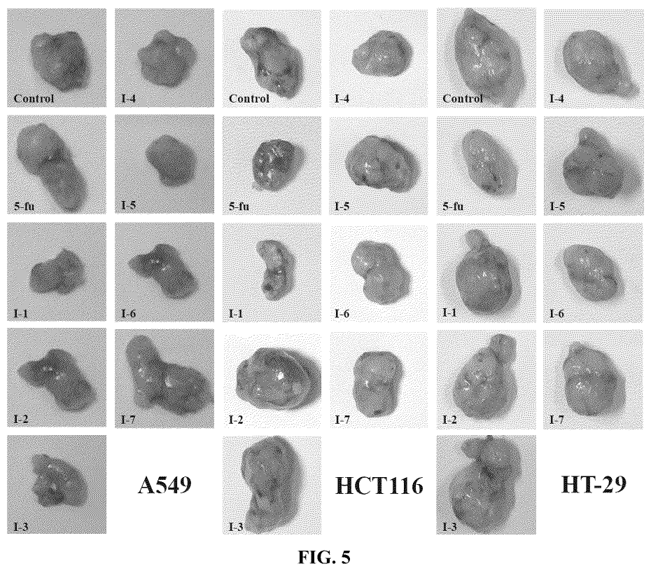

In the HT-29 cell-BALB/c nude mouse subcutaneous tumor transplantation experiment, the volume of the test group with compound I-6, 559.37 mm³, was smaller than that of the control group. After subsequent treatment with capsaicin derivatives, it can be seen that the average tumor weight (FIG. 4) and photographs (FIG. 5) of the tumors showed a correlation between the tumor volume, weight, and photographs. Generally, the stronger the inhibition effect, the smaller the tumor volume and weight are.

In the A549 cell-BALB/c nude mouse subcutaneous tumor transplantation experiment, the inhibitory effects of the seven compounds were all lower than that of 5-fu (43.11%). Among them, compounds I-1 (33.25%), 1-6 (35.19%) and I-7 (33.56%) showed the best inhibitory effects, and compound I-4 (22.05%) also exhibited a certain inhibitory effect;

In the HCT116 cell-BALB/c nude mouse subcutaneous tumor transplantation experiment, the inhibitory effects of the compounds of the present disclosure were all lower than that of 5-fu (44.81%). Among them, compound I-1 (35.13%) exhibited the best inhibitory effect, and compound I-4 (29.86%) also showed a certain inhibitory effect;

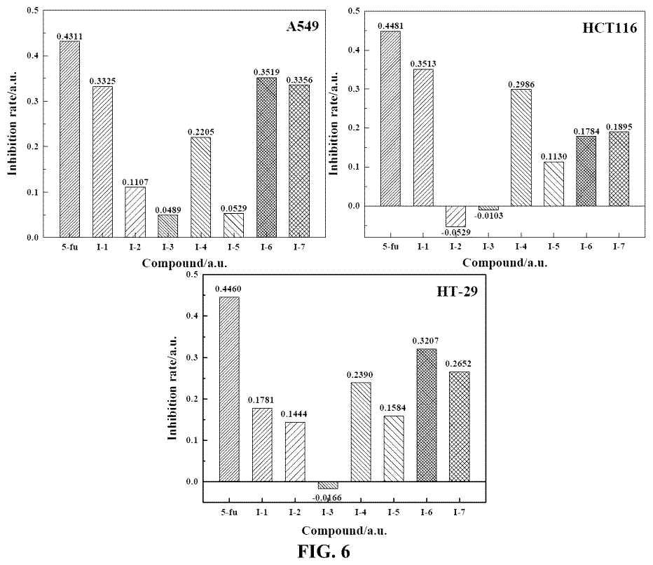

In the HT-29 cell-BALB/c nude mouse subcutaneous tumor transplantation experiment, the inhibitory effects of the compounds of the present disclosure were lower than that of 5-fu (44.60%). Among them, compound I-6 (32.07%) showed the strongest inhibition, while compounds I-4 (23.90%) and I-7 (26.52%) also exhibited certain inhibitory effects. The results are shown in FIG. 6.

1. A compound with anti-tumor activity, having the following structural formula: 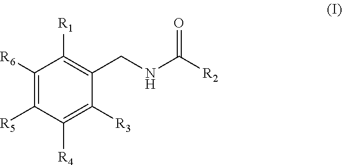
wherein in the formula,
R₁ is selected from hydroxyl, carboxyl, methyl, methoxy, and hydrogen;
R₂ is selected from vinyl, methyl, monochloromethyl, and phenyl;
R₃ is selected from carbomethoxy, ethoxycarbonyl, propoxycarbonyl, hydroxyl, methyl, and hydrogen;
R₄ is selected from hydrogen, hydroxyl, chloro, methyl, bromo, acetyl, carbomethoxy, and chloroacetamidomethyl;
R₅ is selected from hydroxyl, methyl, and hydrogen;
R₆ is selected from hydroxyl, acrylamidomethyl, acetamidomethyl, chloroacetamidomethyl, methyl, and hydrogen.

2. The compound according to claim 1, wherein the compound is selected from the following compounds:

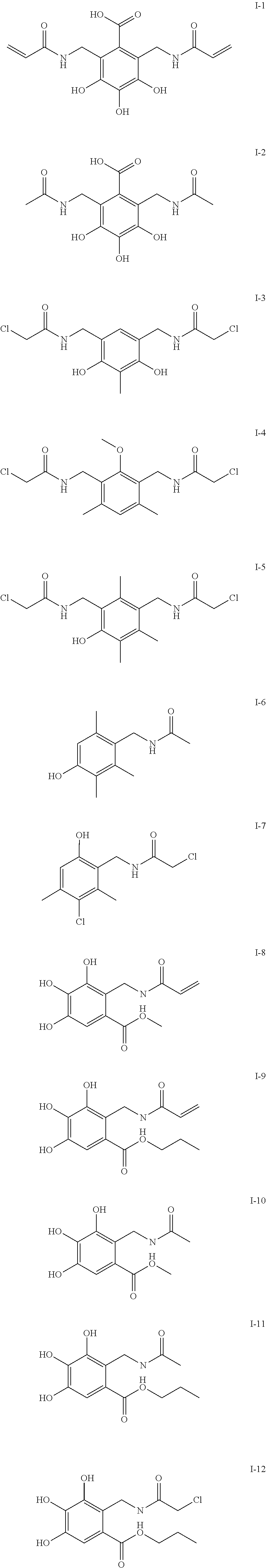

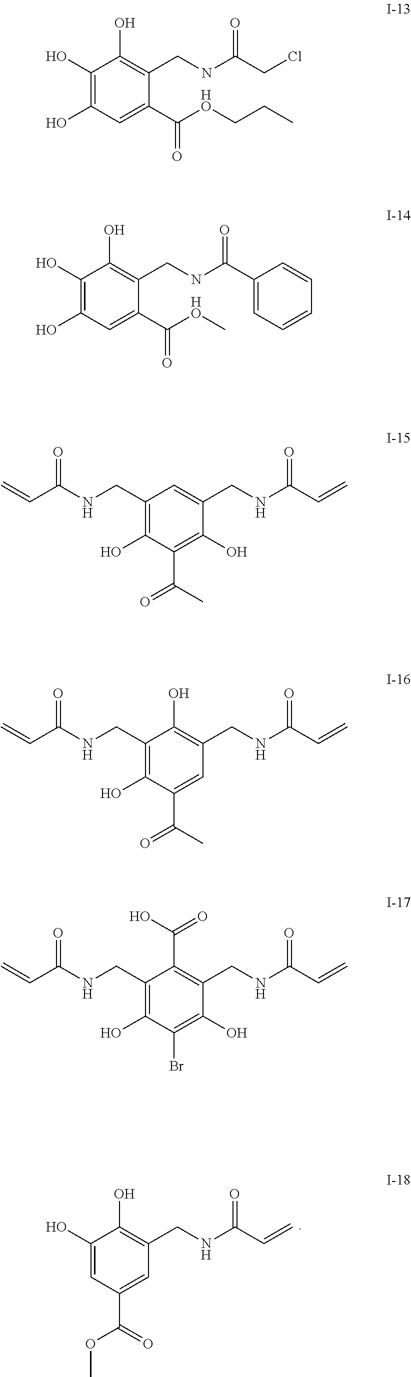

3. A method for preparing the compound according to claim 1-er-2, comprising:
allowing an aromatic compound and an amide compound in a molar ratio of 1:1.2˜2.4 to undergo a substitution reaction in an organic solvent at 25° C. to 55° C. for 48 hours to 96 hours in the presence of a catalyst, in which the molar ratio of the aromatic compound to the catalyst is 1:1.8˜1.9;
then carrying out filtration to obtain a solid product, and
thoroughly washing the solid product with deionized water and drying it to obtain the compound of chemical formula (I).

4. The method according to claim 3, wherein the aromatic compound is gallic acid, methyl gallate, propyl gallate, 2,6-dihydroxytoluene, 3,5-dimethylanisole, 2,3,5-trimethylphenol, 4-chloro-3,5-dimethylphenol, 2,6-dihydroxyacetophenone, 2,4-dihydroxyacetophenone, 4-bromo-3,5-dihydroxybenzoic acid, or methyl 3,4-dihydroxybenzoate.

5. The method according to claim 3, wherein the amide compound is N-hydroxymethyl acrylamide, N-hydroxymethyl acetamide, N-hydroxymethyl chloroacetamide, or N-hydroxymethyl benzamide.

6. The method according to claim 3, wherein the catalyst is concentrated sulfuric acid or anhydrous aluminum trichloride.

7. The method according to claim 3, wherein the solvent is dichloromethane, trichloromethane, acetone, or ethanol.

8. The method according to claim 3, wherein the washed solid product is dried at a temperature of 50° C. to 60° C. for 360 minutes to 420 minutes, to obtain a dried solid product having a water content of 5% by weight or less.

9. A method for treating cancer, comprising administering the compound of formula (I) according to claim 1 to the subject.

10. The method according to claim 9, wherein the cancer is lung cancer, liver cancer, colon cancer, leukemia, cervical cancer, or breast cancer.
